(19) United States
(12) Patent Application Publication
(10) Pub. No.: US 2020/0031858 A1
(43) Pub. Date: Jan. 30, 2020

(54) AVERMECTIN DERIVATIVES AS FXR MODULATORS

(71) Applicant: Hepagene Therapeutics, Inc., Tortola (VG)

(72) Inventor: XIAODONG XU, Doylestown, PA (US)

(21) Appl. No.: 16/500,974

(22) PCT Filed: Apr. 4, 2018
(86) PCT No.: PCT/IB2018/052337

§ 371 Date: Oct. 4, 2019

Related U.S. Application Data
(60) Provisional application No. 62/488,455, filed on Apr. 21, 2017.

(30) Foreign Application Priority Data
Apr. 6, 2017 (CN) 201710223240.1

(51) Int. Cl. C07H 17/08

(52) U.S. Cl. CPC C07H 17/08

(57) ABSTRACT

The present technology is directed to compounds (e.g., Avermectin derivatives), compositions, and methods related to modulation of FXR. In particular the present compounds and compositions may be used to treat FXR-mediated disorders and conditions, including, e.g., liver disease, hyperlipidemia, hypercholesteremia, obesity, metabolic syndrome, cardiovascular disease, gastrointestinal disease, and atherosclerosis, and renal disease.

### FIELD

The present technology is directed to compounds, compositions, and methods related to modulation of farnesoid X receptor (FXR). In particular the present compounds and compositions may be used to treat FXR-mediated disorders and conditions, including, e.g., liver disease, hyperlipidemia, hypercholesteremia, obesity, metabolic syndrome, cardiovascular disease, gastrointestinal disease, atherosclerosis, and renal disease.

### BACKGROUND

Avermectin and its derivatives are used as anthelmintics in humans and as pesticides. Avermectin B1a, which has the structure and numbering shown below, is representative.

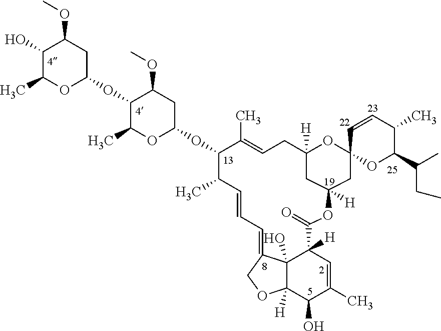 [SMILES: CCC(C)[C@H]1O[C@]2(C=C[C@@H]1C)C[C@@H]1C[C@@H](C/C=C(\C)[C@@H](O[C@H]3C[C@H](OC)[C@@H](O[C@H]4C[C@H](OC)[C@@H](O)[C@H](C)O4)[C@H](C)O3)[C@@H](C)/C=C/C=C3\CO[C@@H]4[C@H](O)C(C)=C[C@@H](C(=O)O1)[C@]34O)O2]

It is a complex glycosylated macrolide incorporating a spirocyclic bis-pyran and a hexahydrobenzofuran into its cyclic skeleton. An exocyclic disaccharide is appended at position 13.

### SUMMARY

In one aspect, the present technology provides avermectin derivatives for use in treating FXR mediated disorders. The avermectin derivatives include a compound according to formula I,

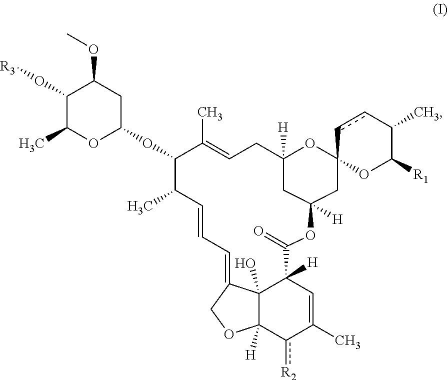

stereoisomers thereof, and/or salts thereof, wherein
R₁ is a substituted or unsubstituted cyclohexyl or C₃-C₄ alkyl group;
R₂ is

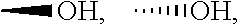

NH₂, NR₄R₅, NNHR₄, or NOR₄;
R₃ is H or

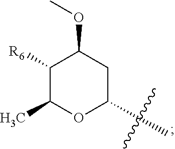

R₄ and R₅ are independently H or a substituted or unsubstituted alkyl, cycloalkyl, alkenyl, aralkyl or heteroaralkyl group;
R₆ is OH, NH₂, NR₇R₈, NR₇C(O)R₉;
R₇ and R₈ are independently H or a substituted or unsubstituted alkyl or alkenyl group;
R⁹ is H, OR¹⁰, or a substituted or unsubstituted alkyl, alkenyl, or aralkyl, group;
R¹⁰ is an unsubstituted alkyl or aralkyl group; and
each [symbol] indicates a single or double bond;
provided that when R₂ is

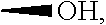

then R₃ is not

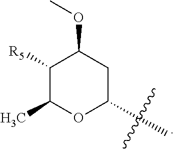

In a related aspect, a composition is provided that includes the compound of formula I or any other compound disclosed herein (including, but not limited to compounds of Formulae IA, IB, IC, and ID) and a pharmaceutically acceptable carrier.

In another aspect, a pharmaceutical composition is provided, the pharmaceutical composition including an effective amount of the compound of any one of the embodiments disclosed herein for treating an FXR-mediated disorder or condition.

In another aspect, a method is provided that includes administering an effective amount of a compound of any one of the embodiments disclosed herein, or administering a pharmaceutical composition including an effective amount of a compound of any one of the disclosed embodiments, to a subject suffering from an FXR-mediated disorder or condition.

In another aspect, a method is provided that includes modulating FXR by contacting FXR with an effective amount of a compound as described herein, including but not limited to any one of the compounds of Formulae I, IA, IB, IC, or ID as described herein.

### DETAILED DESCRIPTION

In various aspects, the present technology provides compounds and methods for modulating FXR and the treatment of FXR-mediated disorders and conditions. The compounds provided herein can be formulated into pharmaceutical compositions and medicaments that are useful in the disclosed methods. Also provided is the use of the compounds in preparing pharmaceutical formulations and medicaments.

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); CF₃; hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; amines; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, oxadiazolonyl (including 1,2,4-oxazol-5(4H)-one-3-yl), isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl,azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The phrase “selectively modulates” as used herein will be understood by persons of ordinary skill in the art and will vary to some extent depending upon the context in which the phrase is used. If there are uses of the phrase which are not clear to persons of ordinary skill in the art, given the context in which the phrase is used, the phrase at minimum refers to the compounds acting through a specific mechanism of action, resulting in fewer off-target effects because the compounds target a particular receptor over other receptors, such as an FXR over a GR receptor, LXR, PPARγ, TGR5 or PXR. This phrase may further be modified as discussed herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, guanidines may exhibit the following isomeric forms in protic organic solution, also referred to as tautomers of each other:

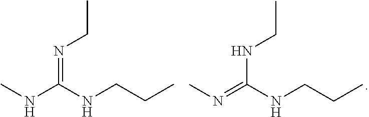

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

In one aspect, the present technology provides avermectin derivatives that modulate FXR and intermediates for making such compounds. For example, there are provided compounds according to formula I:

A compound of Formula I,

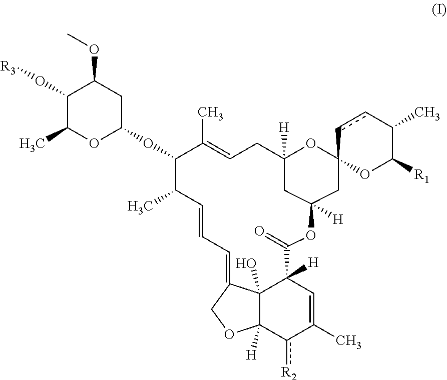

stereoisomers thereof, and/or salts thereof, wherein
R₁ is a substituted or unsubstituted cyclohexyl or C₃-C₄ alkyl group;
R₂ is

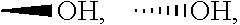

NH₂, NR₄R₅, NNHR₄, or NOR₄;
R₃ is H or

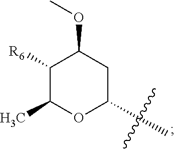

R₄ and R₅ are independently H or a substituted or unsubstituted alkyl, cycloalkyl, alkenyl, aralkyl or heteroaralkyl group;
R₆ is OH, NH₂, NR₇R₈, NR₇C(O)R₉;
R₇ and R₈ are independently H or a substituted or unsubstituted alkyl or alkenyl group;
R⁹ is H, OR¹⁰, or a substituted or unsubstituted alkyl, alkenyl, or aralkyl, group;
R¹⁰ is an unsubstituted alkyl or aralkyl group; and
each [symbol] indicates a single or double bond.

In some embodiments of compounds of Formula I, when R₂ is

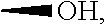

then R₃ is not

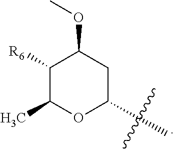

In other embodiments of compounds of Formula I,
R₁ is a substituted or unsubstituted cyclohexyl or C₃-C₄ alkyl group;

R₂ is

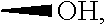

NNHR₄, or NOR₄;
R₃ is H or

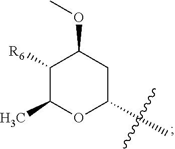

R₄ is H or a substituted or unsubstituted alkyl, cycloalkyl, alkenyl, aralkyl or heteroaralkyl group;
R₆ is OH, NH₂, NR₇R₈, NR₇C(O)R₉;
R₇ and R₈ are independently H or a substituted or unsubstituted alkyl or alkenyl group;
R⁹ is H, OR¹⁰, or a substituted or unsubstituted alkyl, alkenyl, or aralkyl group;
R¹⁰ is an unsubstituted alkyl or aralkyl group; and
each [symbol] independently indicates a single or double bond;
provided that when R₂ is

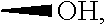

then R₃ is not

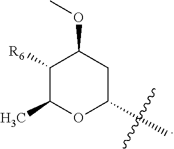

In still other embodiments of compounds of Formula I,
R₂ is

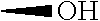

### or NOR₄;

R₄ is H or an unsubstituted alkyl or cycloalkyl group;
R₆ is OH, NH₂, NR₇R₈, NR₇C(O)R₉; and
R₇ and R₈ are independently H or a substituted or unsubstituted alkyl group.

As described in Formula I, R₁ is an unsubstituted cyclohexyl or C₃-C₄ alkyl group. In some embodiments of the compound of Formula I, R₁ is unsubstituted cyclohexyl. In other embodiments, R₁ is an isopropyl group or an isobutyl group.

Compounds of Formula I include those in which R₂ is OH in the naturally occurring stereochemical configuration at position 5, i.e.,

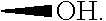

In some embodiments, R₂ has the unnatural configuration,

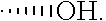

In yet other embodiments, R₂ is NH₂ or NR₄R₅. As noted above, R₄ and R₅ are independently H or a substituted or unsubstituted alkyl, cycloalkyl, alkenyl, aralkyl or heteroaralkyl group, and, e.g., may be independently H, CH₃ or cyclopropyl, among others. In certain embodiments, R₂ is the hydrazine, NNHR₄ or the oxime, NOR₄. In these embodiments, the [symbol] to R₂ is a double bond and the oxime compound of Formula I has the structure of Formula IA.

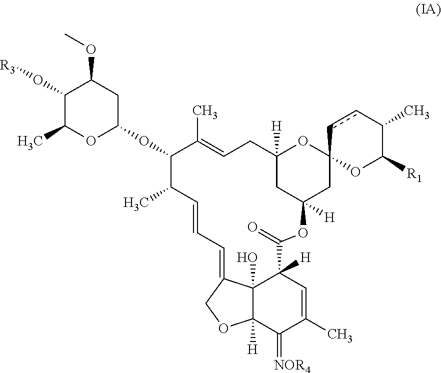

In some embodiments where R₂ is NOR₄, R₄ is H or a substituted or unsubstituted C1-C6 alkyl, benzyl, phenylethyl, or pyridinylmethyl group. For example, R₄ may be H, methyl, ethyl, propyl, cyclopropyl, hydroxymethyl, hydroxyethyl, methoxymethyl, benzyl or pyridinylmethyl. In other embodiments, R₄ is H, methyl, ethyl or propyl.

In some embodiments of compounds of Formula I, R₃ is H. In others it is R₃ is

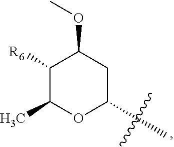

and the compound has the Formula IB.

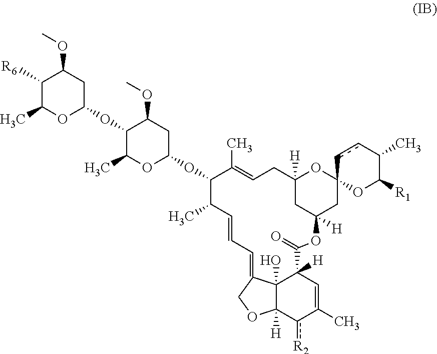

As described above, R₆ can be OH, NH₂, NR₇R₈, NR₇C(O)R₉, and R₇ and R₈ are independently H or a substituted or unsubstituted alkyl or alkenyl group. In some embodiments, R₆ may be OH, NH₂ NHCH₃, N(CH₃)₂, or NHC(O)R₉. In certain embodiments, R₇ and R₈ may independently be H, methyl, or ethyl. In still other embodiments, R₆ is OH, NH₂, or NHC(O)R₉. In some embodiments, R₉ may be H, O-methyl, O-t-butyl, O-fluorenylmethyl, methyl, ethyl, trifluoromethyl, isopropyl, hydroxyethyl, butyl, or methoxymethyl and in others R₉ is H, O-t-butyl, O-fluorenylmethyl, methyl, ethyl, trifluoromethyl, isopropyl, hydroxyethyl, butyl, or methoxymethyl. In some embodiments, R₆ may be OH, NH₂ or NHC(O)CH₃.

In the compounds of Formula I, a double bond may be present between C-22 and C-23. In some embodiments, a single bond is present between C-22 and C-23. Thus, the present technology provides compounds of Formula IC, ID.

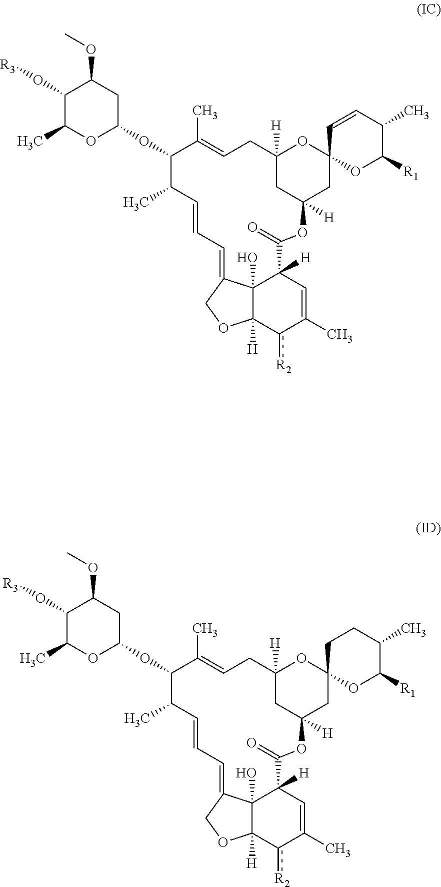

The present technology also provides mixtures of compounds of Formula I. For example, compositions including one or more or two or more compounds of Formula I are provided. In some embodiments the compositions include a compound in which R₁ is an isopropyl group, an isobutyl group, or a cyclohexyl group. The mass ratios of such mixtures may vary, e.g., from 99:1 to 1:99 of a first compound of Formula I to a second compound of Formula I. In some embodiments the ratios may be 99:1, 19:1, 9:1, 5:1, 3:1, 2:1, 1:1, 1:2, 1:3, 1:5, 1:9, 1:19, 1:99 or a range between and including any two of the foregoing values. As a non-limiting example, a mixture of compounds may have a mass ratio of from about 9:1 to about 1:9 in which R₁ is an isopropyl group in the first compound and in which R₁ is an isobutyl group or a cyclohexyl group in the second compound of Formula I. Other mixtures of compounds such as mixtures of stereoisomers at one or more positions or mixtures of compounds having different R₂, R₃, R₄, R₅, R₆, R₇, R₈, and/or R₉ substituents are possible and will be readily appreciated by those of skill in the art.

In a further aspect, a method is provided that includes administering an effective amount of an avermectin or derivative thereof (e.g., a compound as disclosed herein, including but not limited to compounds Formulae I, IA, IB, IC, and ID), or administering a pharmaceutical composition comprising an effective amount of any such compound to a subject suffering from an FXR-mediated disorder or condition. The FXR-mediated disorder or condition may be liver disease, hyperlipidemia, hypercholesteremia, obesity, metabolic syndrome, cardiovascular disease, gastrointestinal disease, atherosclerosis, or renal disease. In some embodiments, the disorder or condition is the disorder or condition may be a liver disease selected from the group consisting of primary biliary cirrhosis (PBC), cerebrotendinous xanthomatosis (CTX), primary sclerosing cholangitis (PSC), nonalcoholic fatty liver disease (NAFLD), nonalcoholic steatohepatitis (NASH), liver fibrosis, and liver cirrhosis.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, the treatment of hyperlipidemia. Another example of an effective amount includes amounts or dosages that are capable of reducing symptoms associated with metabolic syndrome, such as, for example, obesity and/or metabolic syndrome. The effective amount of the compound may selectively modulate FXR. As used herein, a “subject” or “patient” is a mammal, such as a cat, dog, rodent or primate. Typically the subject is a human, and, preferably, a human suffering from or suspected of suffering from an FXR-mediated disorder or condition. The term “subject” and “patient” can be used interchangeably.

In still another aspect, the present technology provides methods of modulating FXR by contacting FXR with an effective amount of any compound as described herein, including but not limited to a compound of Formulae I, IA, IB, IC, or ID.

Thus, the instant present technology provides pharmaceutical compositions and medicaments comprising any of the compounds disclosed herein (e.g., compounds of formulas I, IA, IB, IC, ID) and a pharmaceutically acceptable carrier or one or more excipients or fillers. The compositions may be used in the methods and treatments described herein. Such compositions and medicaments include a therapeutically effective amount of any compound as described herein, including but not limited to a compound of Formulae I, IA, IB, IC, or ID. The pharmaceutical composition may be packaged in unit dosage form.

The pharmaceutical compositions and medicaments may be prepared by mixing one or more compounds of the present technology, stereoisomers thereof, and/or pharmaceutically acceptable salts thereof, with pharmaceutically acceptable carriers, excipients, binders, diluents or the like to prevent and treat disorders associated with the effects of increased plasma and/or hepatic lipid levels. The compounds and compositions described herein may be used to prepare formulations and medicaments that prevent or treat a variety of disorders associated with or mediated by FXR, including but not limited to liver disease, hyperlipidemia, hypercholesteremia, obesity, metabolic syndrome, cardiovascular disease, gastrointestinal disease, atherosclerosis and renal disease. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until for example, (for metabolic syndrome and/or obesity) the elevated plasma or elevated white blood cell count or hepatic cholesterol or triglycerides or progression of the disease state is reduced or stopped. For metabolic syndrome and/or obesity, the progression of the disease state can be assessed using in vivo imaging, as described, or by taking a tissue sample from a patient and observing the target of interest therein.

The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art.

Various assays and model systems can be readily employed to determine the therapeutic effectiveness of the treatment according to the present technology.

Effectiveness of the compositions and methods of the present technology may also be demonstrated by a decrease in the symptoms of hyperlipidemia, such as, for example, a decrease in triglycerides in the blood stream. Effectiveness of the compositions and methods of the present technology may also be demonstrated by a decrease in the signs and symptoms of liver disease, hyperlipidemia, hypercholesteremia, obesity, metabolic syndrome, cardiovascular disease, gastrointestinal disease, atherosclerosis, or renal disease.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The compounds of the present technology can also be administered to a patient along with other conventional therapeutic agents that may be useful in the treatment of liver disease, hyperlipidemia, hypercholesteremia, obesity, metabolic syndrome, cardiovascular disease, gastrointestinal disease, atherosclerosis, or renal disease. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially be effective for the treatment of liver disease, hyperlipidemia, hypercholesteremia, obesity, metabolic syndrome, cardiovascular disease, gastrointestinal disease, atherosclerosis, or renal disease.

In one aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

In another aspect, the present technology provides methods of identifying a target of interest including contacting the target of interest with a detectable or imaging effective quantity of a labeled compound of the present technology. A detectable or imaging effective quantity is a quantity of a labeled compound of the present technology necessary to be detected by the detection method chosen. For example, a detectable quantity can be an administered amount sufficient to enable detection of binding of the labeled compound to a target of interest including, but not limited to, a KOR. Suitable labels are known by those skilled in the art and can include, for example, radioisotopes, radionuclides, isotopes, fluorescent groups, biotin (in conjunction with streptavidin complexation), and chemoluminescent groups. Upon binding of the labeled compound to the target of interest, the target may be isolated, purified and further characterized such as by determining the amino acid sequence.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

The examples herein are provided to illustrate advantages of the present technology and to further assist a person of ordinary skill in the art with preparing or using the compounds of the present technology or salts, pharmaceutical compositions, derivatives, solvates, metabolites, prodrugs, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

### Representative General Synthetic Scheme

Scheme 1 below is the general synthetic scheme showing how to make the compounds of the present technology using procedures known to those of ordinary skill in the art. It is followed by detailed examples of the procedures.

Scheme 1 illustrates generally how oximes of the invention may be made. Starting compound 1 is selectively oxidized with, for example, pyridinium chlorochromate (PCC) in a suitable aprotic organic solvent such as dichloromethane. The resulting ketone is then reacted with hydroxylamine or ether amine in a protic solvent (e.g., water and alcohol such as isopropanol) to provide the desired oxime, 2. One of the saccharide moieties of the compound 2 may be removed using a mixture of mineral acid and organic solvent, e.g., H₂SO₄ and tetrahydrofuran (THF) to give the desired oxime, 4. Alternatively, one of the saccharide moieties of the starting compound 1 may be removed using a mixture of mineral acid and organic solvent, e.g., H₂SO₄ and tetrahydrofuran (THF) to give compound 3. This monosaccharide derivative may then be treated with PCC and hydroxyl- or ether amine to provide the desired oxime, 4.

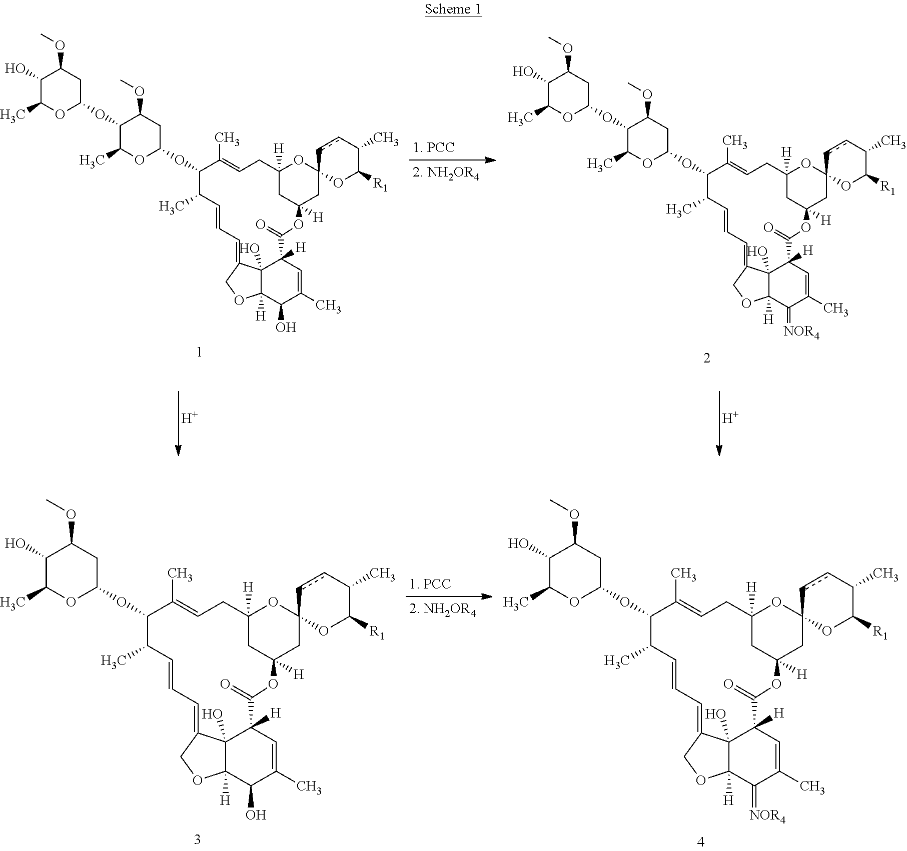

### Example 1—Compound Preparation

### Synthesis of Compound 30

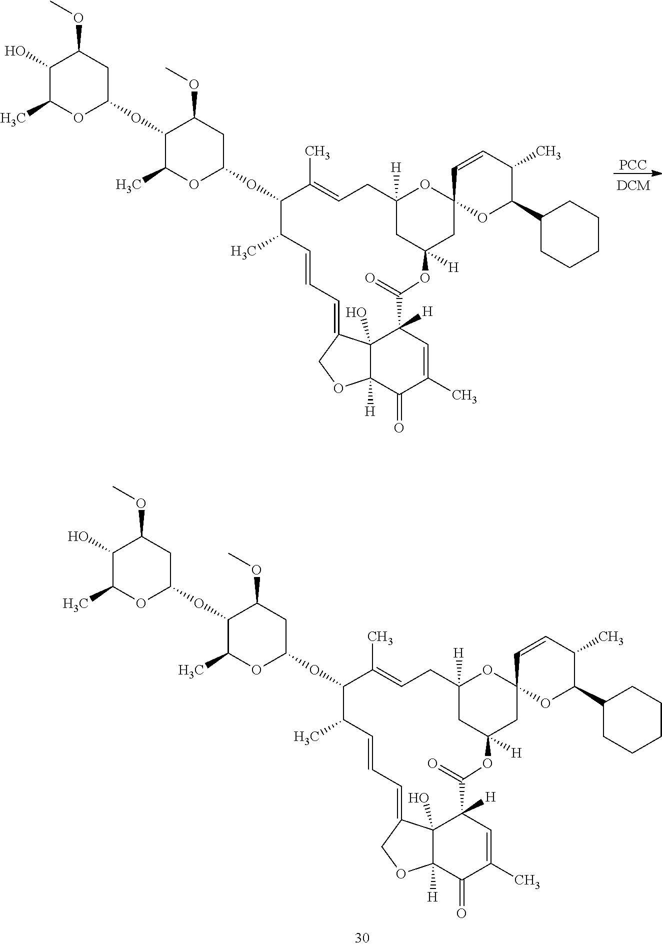 [SMILES: CO[C@H]1C[C@H](O[C@H]2[C@H](C)O[C@@H](O[C@@H]3/C(C)=C/C[C@@H]4C[C@@H](C[C@]5(C=C[C@H](C)[C@@H](C6CCCCC6)O5)O4)OC(=O)[C@@H]4C=C(C)C(=O)[C@H]5OC/C(=C\C=C\[C@@H]3C)[C@]54O)C[C@@H]2OC)O[C@@H](C)[C@@H]1O ; "PCC" ; CO[C@H]1C[C@H](O[C@H]2[C@H](C)O[C@@H](O[C@@H]3/C(C)=C/C[C@@H]4C[C@@H](C[C@]5(C=C[C@H](C)[C@@H](C6CCCCC6)O5)O4)OC(=O)[C@@H]4C=C(C)C(=O)[C@H]5OC/C(=C\C=C\[C@@H]3C)[C@]54O)C[C@@H]2OC)O[C@@H](C)[C@@H]1O]

To a solution of Doramectin (200 mg, 0.2 mmol) in dichloromethane (10 mL) was added silica gel (200 mg) and PCC (72 mg, 0.3 mmol) at 0° C. The system was allowed warm to room temperature and stirred for another 4 h. After filtration, the filtrate was washed with water. The resulted aqueous phase was extracted with 3×30 mL of ethyl acetate. The organic layers were combined and dried over anhydrous magnesium sulfate and concentrated under vacuum. The crude was purified by Prep-HPLC with the following conditions (2#-Analyse HPLC-SHIMADZU(HPLC-10)): Column, XSelect CSH Prep C18 OBD Column, 19×250 mm, 5 m; mobile phase, water (0.05% FA) and ACN (89.0% ACN up to 94.0% in 7 min); Detector, UV 254/220 nm. This resulted in 30.9 mg (15%) of 30 as a white solid. LCMS (ESI, m/z): [M+Na]⁺=919.8. H-NMR (300 MHz, DMSO-d₆, ppm): δ 6.79-6.77 (m, 1H), 5.96-5.67 (m, 5H), 5.54-5.50 (m, 1H), 5.23-5.21 (m, 1H), 5.18-5.12 (m, 1H), 5.03-4.91 (m, 1H), 4.73-4.71 (m, 1H), 4.65-4.58 (m, 2H), 3.90-3.72 (m, 3H), 3.59-3.33 (m, 9H), 3.28-3.08 (m, 4H), 2.91-2.85 (m, 2H), 2.73-2.62 (m, 1H), 2.24-2.00 (m, 6H), 1.89-1.85 (m, 1H), 1.76-1.63 (m, 7H), 1.54-1.20 (m, 10H), 1.33-1.08 (m, 12H), 0.87 (d, J=7.0 Hz, 4H).

### Synthesis of Compound 31

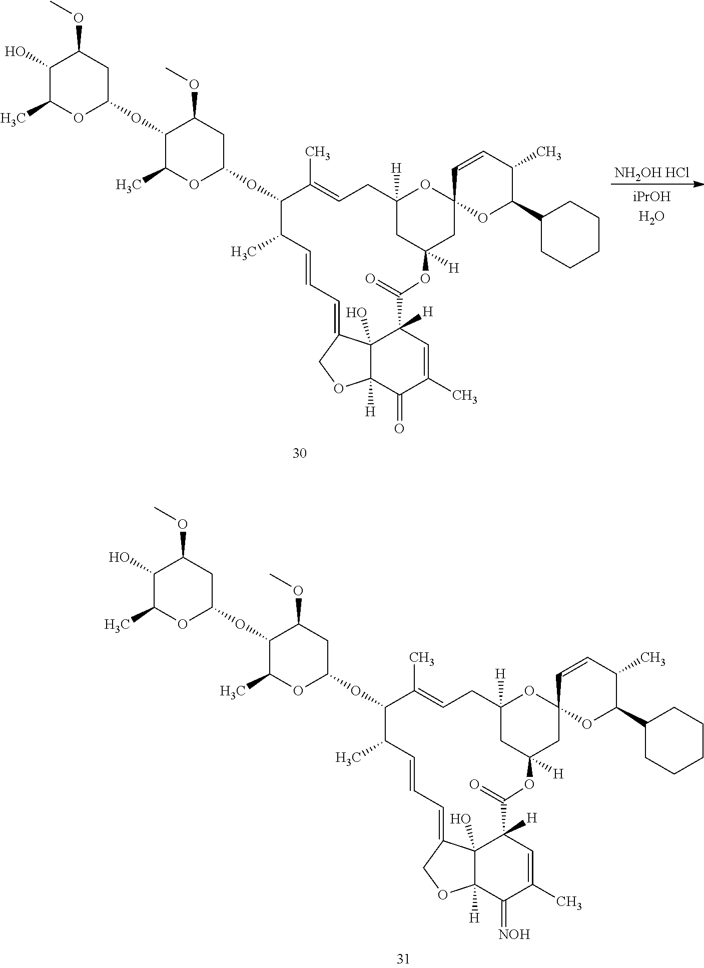

To a solution of 30 (1.1 g, 1.23 mmol) in PrOH (25 mL) and water (4 mL) was added hydroxylamine hydrochloride (1 g, 14.39 mmol). The mixture was stirred for 5 h at room temperature. The resulting mixture was extracted with 3×50 mL of MTBE and the organic layers were combined. The organic phase was washed with water and brine. The residue was concentrated under vacuum after dried over anhydrous magnesium sulfate. The crude product was purified by Prep-HPLC with the following conditions (2#-Analyse HPLC-SHIMADZU(HPLC-10)): Column, SunFire Prep C18 OBD Column, 19×150 mm, 5 μm 10 nm; mobile phase, water (0.1% FA) and ACN (85.0% ACN up to 94.0% in 7 min); Detector, UV 254/220 nm. This resulted in 178 mg (16%) of 31 as a white solid. LCMS (ESI, m/z): [M+Na]⁺=934.8. H-NMR (300 MHz, DMSO-d₆, ppm): δ 11.50 (s, 1H), 5.86-5.77 (m, 3H), 5.74-5.65 (m, 2H), 5.48-5.41 (m, 1H), 5.38-5.22 (m, 1H), 5.21-5.08 (m, 1H), 5.03-4.91 (m, 2H), 4.75-4.72 (m, 1H), 4.45-4.40 (m, 1H), 3.98-3.92 (m, 1H), 3.84-3.69 (m, 2H), 3.64-3.49 (m, 2H), 3.43-3.33 (m, 6H), 3.32-3.05 (m, 4H), 3.04-2.92 (m, 1H), 2.45-2.15 (m, 5H), 2.08-1.97 (m, 1H), 1.94-1.82 (m, 4H), 1.80-1.63 (m, 4H), 1.61-1.45 (m, 7H), 1.43-1.20 (m, 3H), 1.20-1.05 (m, 12H), 1.02-0.96 (m, 3H), 0.91-0.87 (m, 1H).

### Synthesis of Compound 32

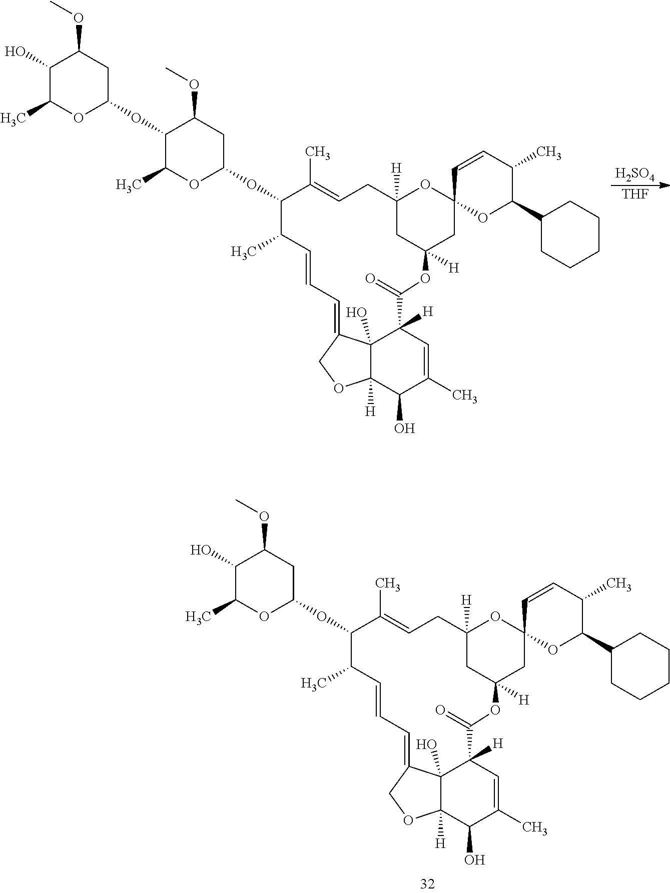

To a solution of H₂SO₄ (0.92 mL, 50%) in THF (3.7 mL) was added Doramectin (222 mg, 0.25 mmol) at 0° C. The mixture was stirred for 16 h at room temperature. The reaction was quenched by the addition of 50 mL of water. The resulting solution was extracted with 3×50 mL of ethyl acetate and the organic layers were combined. The organic phase was washed with water and brine. The residue was concentrated under vacuum after dried over anhydrous magnesium sulfate. The crude product was purified by Prep-HPLC with the following conditions (2#-Analyse HPLC-SHIMADZU(HPLC-10)): Column, XSelect CSH Prep C18 OBD Column, 19×250 mm, 5 m; mobile phase, water (0.05% FA) and ACN (70.0% ACN up to 88.0% in 7 min); Detector, UV 254/220 nm. This resulted in 34.1 mg (36%) of 32 as a white solid. LCMS (ESI, m/z): [M+Na]⁺=777.5. H-NMR (300 MHz, DMSO-d₆, ppm): δ 5.83-5.67 (m, 4H), 5.65-5.52 (m, 1H), 5.51-5.50 (m, 1H), 5.49-5.48 (m, 1H), 5.31-5.23 (m, 1H), 5.22-5.09 (m, 1H), 5.06-4.70 (m, 1H), 4.69-4.65 (m, 1H), 4.65-4.63 (m, 1H), 4.62-4.54 (m, 1H), 4.54-4.46 (m, 1H), 3.89-3.61 (m, 2H), 3.60-3.59 (m, 1H), 3.58-3.36 (m, 1H), 3.34-3.30 (m, 4H), 3.24-3.20 (m, 1H), 3.03-3.02 (m, 1H), 2.90-2.89 (m, 1H), 2.89-2.88 (m, 1H), 2.49-2.15 (m, 5H), 2.01-1.66 (m, 8H), 1.66-1.33 (m, 7H), 1.30-1.08 (m, 12H), 0.88-0.86 (m, 3H), 0.78-0.76 (m, 1H).

### Synthesis of Compound 33

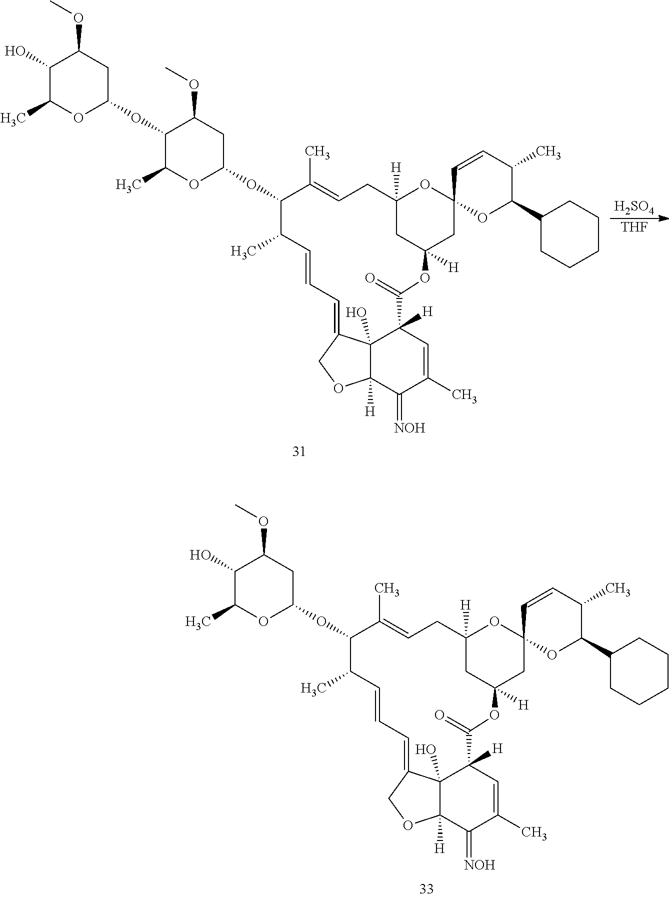

To a solution of sulfuric acid (0.16 mL, 50%) in THF (10 mL) was added 31 (200 mg, 0.22 mmol). The solution was stirred for 6 h at room temperature. The reaction was quenched by the addition of 50 mL of water. The pH value of the solution was adjusted to 8 with saturated sodium bicarbonate solution. The resulted mixture was extracted with ethyl acetate. The organic phase was combined and washed with water and brine. The residue was concentrated under vacuum after dried over anhydrous magnesium sulfate. The crude product was purified by Prep-HPLC with the following conditions (2#-Analyse HPLC-SHIMADZU(HPLC-10)): Column, SunFire Prep C18 OBD Column, 19×150 mm, 5 m 10 nm; Mobile phase, water (0.1% FA) and ACN (70.0% ACN up to 90.0% in 7 min); Detector, UV 254/220 nm. Then the product was obtained and concentrated under vacuum. This resulted in 13.8 mg (8%) of 33 as a white solid. LCMS (ESI, m/z): [M+H]⁺=768.6. H-NMR (300 MHz, DMSO-d₆, ppm): δ 11.50 (s, 1H), 5.86-5.77 (m, 2H), 5.74-5.65 (m, 2H), 5.61-5.53 (m, 1H), 5.48-5.41 (m, 1H), 5.17-5.08 (m, 2H), 5.03-4.95 (m, 1H), 4.73-4.68 (m, 2H), 4.92-4.78 (m, 2H), 4.75-4.68 (m, 1H), 3.28-3.12 (m, 2H), 2.98-2.87 (m, 2H), 2.60-2.51 (m, 1H), 2.25-2.15 (m, 4H), 2.08-1.97 (m, 1H), 1.94-1.82 (m, 4H), 1.80-1.63 (m, 4H), 1.61-1.45 (m, 7H), 1.45-1.33 (m, 5H), 1.32-1.21 (m, 4H), 1.15-1.02 (m, 7H), 0.99-0.93 (m, 3H), 0.91-0.87 (m, 1H).

### Synthesis of Compound 34

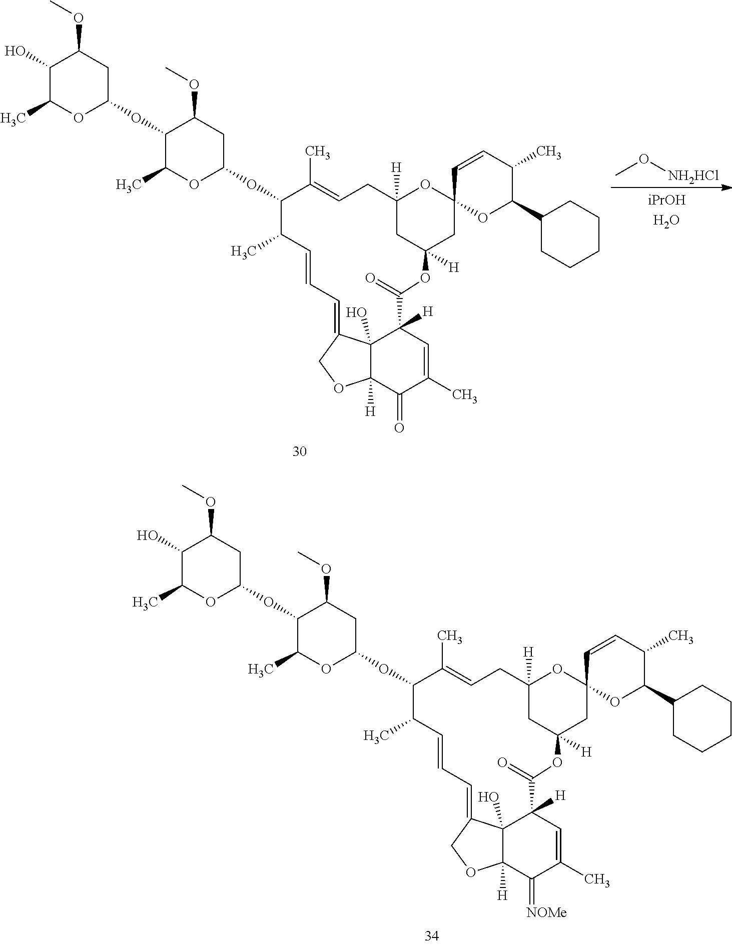

To a solution of 30 (200 mg, 0.22 mmol) in iPrOH (5 mL) and water (0.8 mL) was added O-methylhydroxylamine hydrochloride (200 mg, 2.39 mmol). The solution was stirred for 4 h at room temperature. The resulting solution was extracted with 3×75 mL of ethyl acetate and the organic layers were combined. The organic phase was washed with water and brine. The residue was concentrated under vacuum after dried over anhydrous magnesium sulfate. The crude product was purified by Prep-HPLC with the following conditions (2 #-Analyse HPLC-SHIMADZU(HPLC-10)): Column, SunFire Prep C18 OBD Column, 19×150 mm, 5 m 10 nm; Mobile phase, water (0.1% FA) and ACN (92.0% ACN up to 95.0% in 7 min); Detector, UV 254/220 nm. Then the product was obtained and concentrated under vacuum. This resulted in 13.8 mg (8%) of 34 as a white solid. LCMS (ESI, m/z): [M+Na]⁺=948.8. H-NMR (300 MHZ, DMSO-d₆, ppm): δ 5.96-5.77 (m, 2H), 5.76-5.67 (m, 2H), 5.63-5.55 (m, 1H), 5.51-5.43 (m, 1H), 5.37-5.22 (m, 2H), 5.10-5.02 (m, 1H), 5.01-4.98 (m, 1H), 4.92-4.81 (m, 1H), 4.75-4.68 (m, 1H), 4.45-4.31 (m, 2H), 4.21-4.16 (m, 1H), 3.91-3.65 (m, 6H), 3.55-3.46 (m, 2H), 3.32-3.12 (m, 9H), 3.10-3.05 (m, 1H), 2.88-2.75 (m, 1H), 2.25-2.02 (m, 5H), 2.00-1.93 (m, 1H), 1.81-1.75 (m, 4H), 1.72-1.53 (m, 4H), 1.62-1.41 (m, 8H), 1.35-1.26 (m, 3H), 1.25-1.01 (m, 12H), 0.89-0.83 (m, 3H), 0.81-0.77 (m, 1H).

### Synthesis of Compound 35

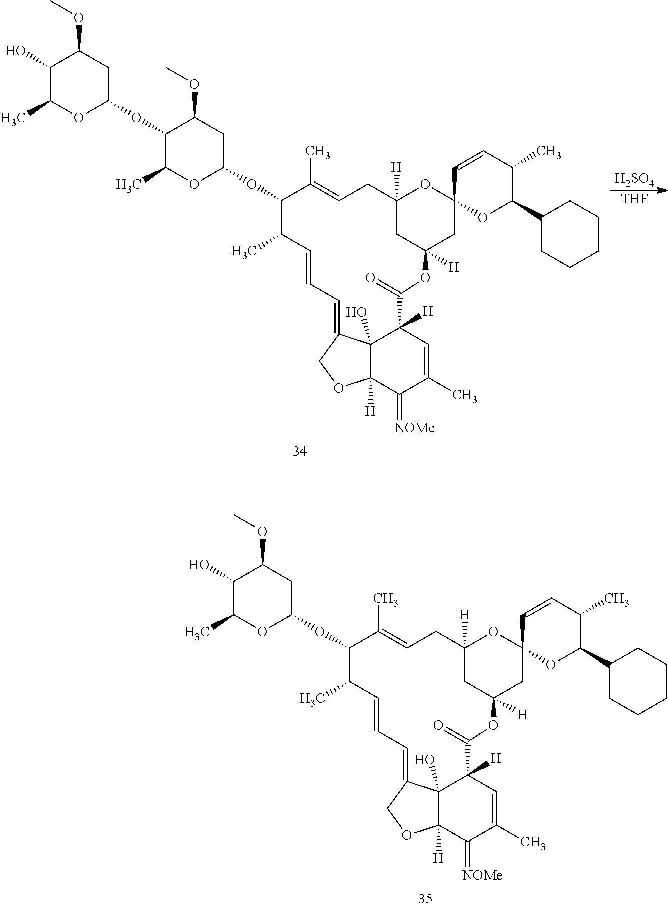

To a solution of sulfuric acid (0.16 mL, 50%) in THF (3.7 mL) was added 34 (200 mg, 0.22 mmol). The resulting solution was stirred for 6 h at room temperature. The reaction was diluted with 50 mL of water. The pH value of the solution was adjusted to 8 with saturated sodium bicarbonate solution. The resulting mixture was extracted with 3×75 mL of ethyl acetate and the organic layers were combined. The organic phase was washed with water and brine. The residue was concentrated under vacuum after dried over anhydrous magnesium sulfate. The crude product was purified by Prep-HPLC with the following conditions (2#-Analyse HPLC-SHIMADZU(HPLC-10)): Column, SunFire Prep C18 OBD Column, 19×150 mm, 5 m 10 nm; mobile phase, water (0.1% FA) and ACN (87.0% ACN up to 90.0% in 7 min); Detector, UV 254/220 nm. Then the product was obtained and concentrated under vacuum. This resulted in 42.1 mg (25%) of 35 as a white solid. LCMS (ESI, m/z): [M+H]⁺=782.7. H-NMR (300 MHz, DMSO-d₆, ppm): δ 5.86-5.77 (m, 3H), 5.76-5.67 (m, 2H), 5.63-5.55 (m, 1H), 5.51-5.43 (m, 1H), 5.15-5.05 (m, 1H), 5.01-4.92 (m, 1H), 4.75-4.68 (m, 1H), 4.65-4.55 (m, 2H), 4.40-4.31 (m, 1H), 3.97-3.75 (m, 5H), 3.65-3.56 (m, 1H), 3.32-3.22 (m, 2H), 2.95-2.88 (m, 1H), 2.67-2.58 (m, 1H), 2.28-2.05 (m, 4H), 2.04-2.00 (m, 1H), 1.85-1.75 (m, 4H), 1.72-1.68 (m, 2H), 1.67-1.63 (m, 2H), 1.55-1.44 (m, 7H), 1.41-1.01 (m, 14H), 0.89-0.67 (m, 5H).

### Example 2—Biological Assay

The compounds of the present technology may be assayed using the following procedure and will be shown to have FXR binding activity.

### FXR Coactivator Assay

### Reagents:

### LanthaScreen™ TR-FRET Farnesoid X Receptor Coactivator Assay

GW4064 as a positive control

### Process:

1. All of compounds were 3-fold serial diluted from 10 mM stock for 10 doses in DMSO.
2. Dilute each 100× agonist serial dilution to 2× using Complete Coregulator buffer G.
3. Transfer 10 μl of each of the 2× agonist serial dilutions to 384 well assay plates.
4. Add 5 μl of 4×FXR-LBD to 384 well assay plates.
5. Add 5 μl of 4× peptide/4× antibody solution to 384 well assay plates.
6. Incubate at room temperature protected from light.
7. Read the plate at wavelengths of 520 nm and 495 nm on Envision 2104 plate reader.
8. Calculate the TR-FRET ratio by dividing the emission signal at 520 nm by the emission signal at 495 nm.
9. Calculate EC50 by fitting % Activity values and log of compound concentrations to nonlinear regression (dose response−variable slope) with Graphpad 5.0.

TABLE 1
FXR Coactivator Assay

| | EC50 |
|---|---|
| | FXR Coactivator |
| Compound | Assay |
| 31 | A |
| 32 | B |
| 33 | A |
| 35 | C |

A = 10 nM to 100 nM
B = 101 nM-300 nM
C = 301 nM-1 uM

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art.

Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

What is claimed is:

1. A compound of Formula I, 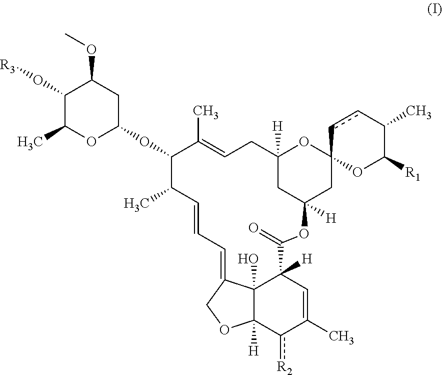
stereoisomers thereof, and/or salts thereof, wherein
R₁ is a substituted or unsubstituted cyclohexyl or C₃-C₄ alkyl group;
R₂ is 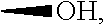
NNHR₄, or NOR₄;
R₃ is H or 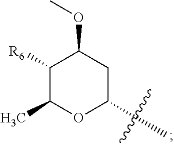
R₄ is H or a substituted or unsubstituted alkyl, cycloalkyl, alkenyl, aralkyl or heteroaralkyl group;
R₆ is OH, NH₂, NR₇R₈, NR₇C(O)R₉;
R₇ and R₈ are independently H or a substituted or unsubstituted alkyl or alkenyl group;
R⁹ is H, OR¹⁰, or a substituted or unsubstituted alkyl, alkenyl, or aralkyl group;
R¹⁰ is an unsubstituted alkyl or aralkyl group; and
each [symbol] independently indicates a single or double bond;
provided that when R₂ is 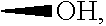
then R₃ is not

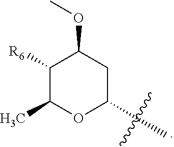

2. The compound of claim 1 wherein
R₂ is 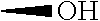
or NOR₄;
R₄ is H or an unsubstituted alkyl or cycloalkyl group;
R₆ is OH, NH₂, NR₇R₈, NR₇C(O)R₉; and
R₇ and R₈ are independently H or a substituted or unsubstituted alkyl group.

3. The compound of claim 1 or claim 2 wherein R₁ is a cyclohexyl group, isopropyl group or an isobutyl.

4. The compound of any one of claims 1-3 wherein R₂ is

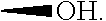

5. The compound of any one of claims 1-3 wherein R₂ is NOR₄.

6. The compound of any one of claims 1-5 wherein R₄ is H, methyl, ethyl or propyl.

7. The compound of any one of claims 1-6 wherein R₃ is H.

8. The compound of any one of claims 1-6 wherein R₃ is

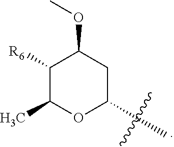

9. The compound of claim 8 wherein R₆ is OH, NH₂, or NHC(O)R₉.

10. The compound of claim 9 wherein R₉ is H, O-t-butyl, O-fluorenylmethyl, methyl, ethyl, trifluoromethyl, isopropyl, hydroxyethyl, butyl, or methoxymethyl.

11. The compound of any one of claims 1-10 wherein a double bond is present between C-22 and C-23.

12. The compound of any one of claims 1-10 wherein a single bond is present between C-22 and C-23.

13. A composition comprising one or more compounds of any one of claims 1-12.

14. The composition of claim 13 comprising a pharmaceutically acceptable carrier.

15. A pharmaceutical composition comprising an effective amount of the compound of any one of claims 1-14 for treating an FXR-mediated disorder or condition.

16. The pharmaceutical composition of claim 15 wherein the disorder or condition is selected from the group consisting of liver disease, hyperlipidemia, hypercholesteremia, obesity, metabolic syndrome, cardiovascular disease, gastrointestinal disease, atherosclerosis, and renal disease.

17. The pharmaceutical composition of claim 15 wherein the disorder or condition is a liver disease selected from the group consisting of primary biliary cirrhosis (PBC), cerebrotendinous xanthomatosis (CTX), primary sclerosing cholangitis (PSC), nonalcoholic fatty liver disease (NAFLD), nonalcoholic steatohepatitis (NASH), liver fibrosis, and liver cirrhosis.

18. A method of treatment comprising administering an effective amount of a compound of any one of claims 1-12, or administering a pharmaceutical composition comprising an effective amount of a compound of any one of claims 1-12, to a subject suffering from an FXR-mediated disorder or condition.

19. The method of claim 18, wherein the disorder or condition is selected from the group consisting of liver disease, hyperlipidemia, hypercholesteremia, obesity, metabolic syndrome, cardiovascular disease, gastrointestinal disease, atherosclerosis, and renal disease.

20. The method of claim 18, wherein the disorder or condition is a liver disease selected from the group consisting of primary biliary cirrhosis (PBC), cerebrotendinous xanthomatosis (CTX), primary sclerosing cholangitis (PSC), nonalcoholic fatty liver disease (NAFLD), nonalcoholic steatohepatitis (NASH), liver fibrosis, and liver cirrhosis.

21. A method comprising modulating FXR by contacting FXR with an effective amount of a compound of any one of claims 1-12.
